(19) United States
(12) Plant Patent Application Publication
(10) Pub. No.: US 2026/0083037 P1
(43) Pub. Date: Mar. 19, 2026

(54) PHILODENDRON PLANT NAMED “GREEN DREAMS PHILODENDRON”

(50) ;

(71) Applicant: Robert Roe, Umatilla, FL (US)

(72) Inventor: Robert Roe, Umatilla, FL (US)

(21) Appl. No.: 18/889,314

(22) Filed: Sep. 18, 2024

(51) Int. Cl. A01H 6/10; A01H 5/04; A01H 5/12

(52) U.S. Cl. CPC A01H 6/10; A01H 5/04; A01H 5/12
USPC PLT/381

(57) ABSTRACT

A new and distinct cultivar named “Green Dreams Philodendron”.

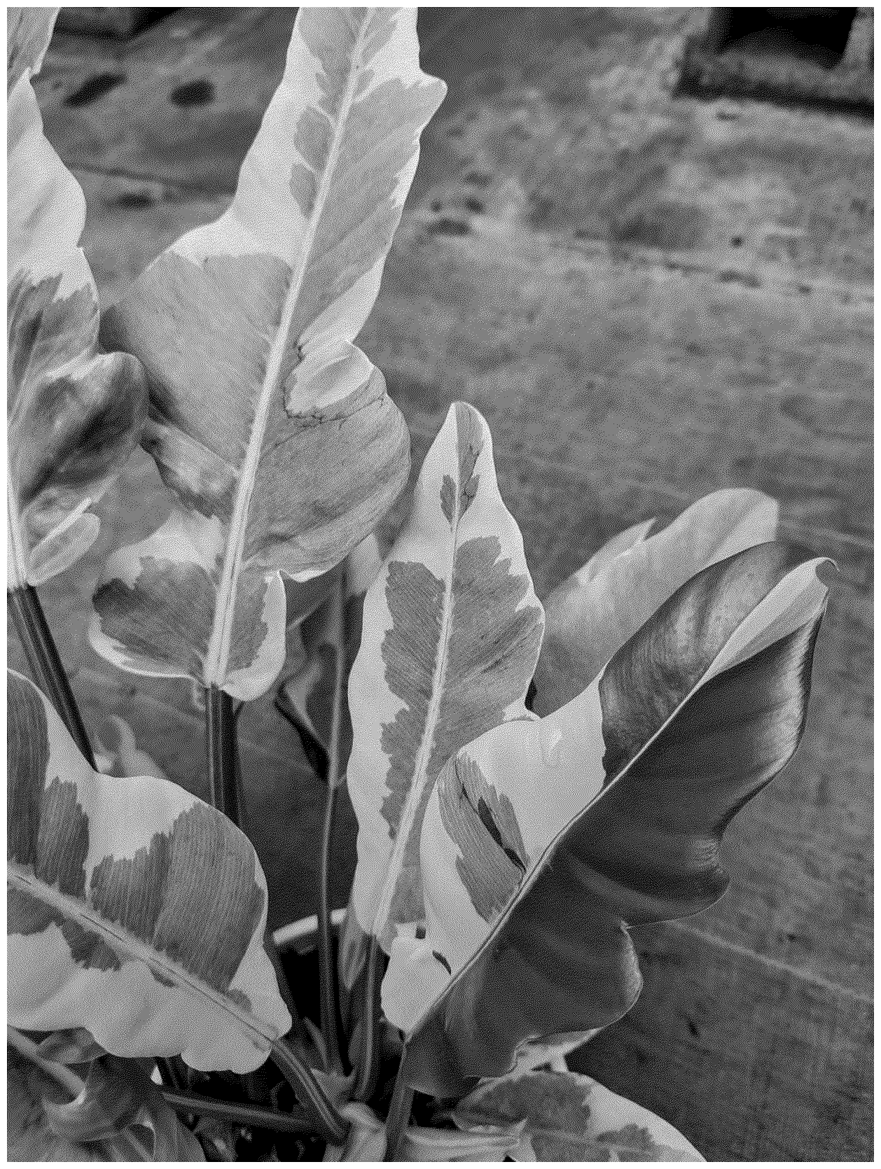

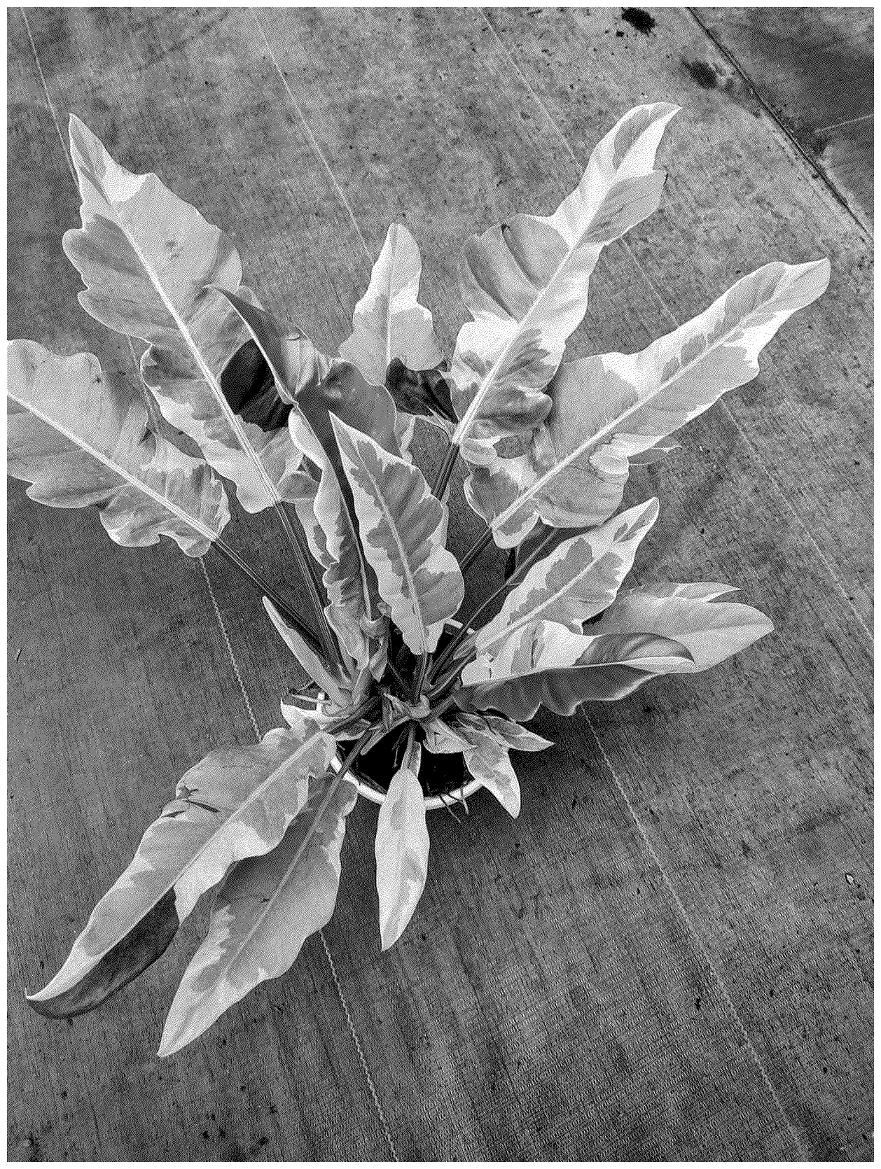

### CROSS-REFERENCE TO RELATED APPLICATIONS

The present application claims priority to U.S. Provisional Application No. 63/538,970 filed Sep. 18, 2023, titled “PHILODENDRON PLANT NAMED “GREEN DREAMS PHILODENDRON” which is hereby incorporated by reference in its entirety.

### BACKGROUND

The present invention relates to a new and distinct cultivar of Philodendron referred to as “Green Dreams Philodendron” plant.

The new Green Dreams Philodendron is a product of an accidental breeding by the Inventor in Umatilla, Florida.

### SUMMARY OF THE INVENTION

The new Green Dreams Philodendron has not been observed under all possible environmental conditions. The phenotype may vary somewhat with variations in environment such as temperature, light intensity, fertilizer rate, and/or irrigation amount and frequency without any variance in genotype.

The following traits have been repeatedly observed and are determined to be the unique characteristics of “Green Dreams Philodendron.” These characteristics in combination distinguish “Green Dreams Philodendron” as a new and distinct cultivar.
1. Plants of the new Green Dreams Philodendron have lime green leaves with dark green variegation.
2. Leaves can grow 8-inches to 10-inches long.
3. Stems are dark green to light green and upright.

### DETAILED BOTANICAL DESCRIPTION

The following observations, measurements, and comparisons describe plants grown in Umatilla, Florida, under a polycarbonate-covered greenhouse and conditions which closely approximate those used in general, standard commercial horticultural practice. Plants were grown in liquid granular fertilized in a dry tropical environment. Light level ranged from 2000 to 2,200 footcandles.

What is claimed is:

1. A new and distinct cultivar named “Green Dreams Philodendron” as illustrated and described.
